(19) United States
(12) Patent Application Publication
(10) Pub. No.: US 2023/0210069 A1
(43) Pub. Date: Jul. 6, 2023

(54) PLANT GROWTH MANAGEMENT SYSTEM USING MOVABLE FLOWERPOT AND CONTROL METHOD USING THE SAME

(71) Applicant: Anthony Sunbeom KIM, Seoul (KR)

(72) Inventor: Anthony Sunbeom KIM, Seoul (KR)

(21) Appl. No.: 17/646,534

(22) Filed: Dec. 30, 2021

(51) Int. Cl. A01G 27/00; A01G 9/08; A01G 9/26; G01N 33/24; H02J 50/10; H02J 50/90; G05D 3/10

(52) U.S. Cl. CPC A01G 27/003; A01G 9/088; A01G 27/008; A01G 9/26; G01N 33/246; H02J 50/10; H02J 50/90; G05D 3/105

(57) ABSTRACT

Disclosed are a plant growth management system and a control method thereof. A station device of the plant growth management system includes a flowerpot detector configured detect entry of a movable flowerpot device and to generate a detection signal according to whether entry of the movable flowerpot device is detected, a wireless charger configured to wirelessly supply power to the movable flowerpot device according to the detection signal, and a water supply device configured to supply water to a flowerpot included in the movable flowerpot device according to the detection signal, wherein the movable flowerpot device includes an obstacle detection sensor and a solar detection sensor, performs autonomous driving based on the obstacle detection sensor and the solar detection sensor, searches for a space, an illuminance value of which is equal to or greater than a preset illuminance value, through the autonomous driving, and performs rotational motion in the searched space.

### BACKGROUND OF THE DISCLOSURE

### Field of the Disclosure

The present invention relates to a plant growth management system, and more particularly to a plant growth management system including a movable flowerpot device and a station device.

### Description of the Related Art

In general, plants grown indoors for aesthetics and air purification are planted in pots and placed in a sunny location to grow plants.

At this time, if a flowerpot is placed in a location where sunlight is well received, there is a problem in that the plant bends in a direction in which sunlight enters, and in the case of expensive bonsai, decorativeness is degraded compared to investment cost.

In addition, in the case of a plant planted in pots, only a part of the plant, which is directed toward sunlight, receives sunlight, and thus in the case of other parts that are not directed toward sunlight, there is a problem that the plant easily dies, and accordingly, a user needs to search for a way to artificially turn the flowerpot at regular time intervals, but this is disadvantageously cumbersome.

### CITED REFERENCE

### Patent Document

Korean Patent Publication No. 10-1557679, “Integral Swivel Planter Stand”

### SUMMARY OF THE INVENTION

Therefore, the present invention has been made in view of the above problems, and it is an object of the present invention to provide technology for autonomously changing the position of a flowerpot to automatically adjust the amount of sunlight that a plant is capable of receiving and to prevent damage to the plant due to movement of the flowerpot, thereby improving user convenience.

It is another object of the present invention to provide technology for providing wireless charging and automatic water supply, thereby stably supplying power and inducing successful growth of plants.

It is yet another object of the present invention to provide technology for selectively supplying water to a flowerpot, thereby minimizing damage to plants due to moisture supply.

In accordance with an aspect of the present invention, the above and other objects can be accomplished by the provision of a station device of a plant growth management system, comprising: a flowerpot detector configured detect entry of a movable flowerpot device and to generate a detection signal according to whether entry of the movable flowerpot device is detected; a wireless charger configured to wirelessly supply power to the movable flowerpot device according to the detection signal; and a water supply device configured to supply water to a flowerpot included in the movable flowerpot device according to the detection signal, wherein the movable flowerpot device includes an obstacle detection sensor and a solar detection sensor, performs autonomous driving based on the obstacle detection sensor and the solar detection sensor, searches for a space, an illuminance value of which is equal to or greater than a preset illuminance value, through the autonomous driving, and performs rotational motion in the searched space.

In accordance with an aspect, the wireless charger may include a transmitting coil configured to wirelessly supply power to a receiving coil included in the movable flowerpot device; and a detection coil configured to detect misalignment between the transmitting coil and the receiving coil.

In accordance with an aspect, the water supply device may include a water nozzle configured to supply water to the flowerpot; a camera installed in the water nozzle and configured to detect a position of a plant included in the flowerpot; and a nozzle driver configured to adjust a position of the water nozzle based on the position of the plant detected through the camera.

In accordance with an aspect, the movable flowerpot device may determine whether to move to the station device according to time information output from a real time clock (RTC) module.

In accordance with an aspect, the movable flowerpot device may monitor at least one of a state of charge of a battery and moisture content of soil in the flowerpot and may determine whether to move to the station device according to a monitoring result.

In accordance with an aspect, when an illuminance value detected through the solar detection sensor is equal to or less than the preset illuminance value in the searched space, the movable flowerpot device may stop the rotational motion, may search for another space, an illuminance value of which is equal to or greater than the preset illuminance value, through the autonomous driving, and may perform the rotational motion in the other searched space.

In accordance with another aspect of the present invention, there is provided a movable flowerpot device of a plant growth management system, comprising: a detector including an obstacle detection sensor and a solar detection sensor, and configured to detect whether an obstacle is present through the obstacle detection sensor and to detect an illuminance value through the solar detection sensor; a controller configured to determine a moving path based on whether the obstacle is present and the detected illuminance value, to control the movable flowerpot device to perform autonomous driving on the moving path, to determine whether the movable flowerpot device moves to a station device, and to control the movable flowerpot device to move to the station device according to a determination result of whether the movable flowerpot device moves to the station device; and a power part configured to wirelessly receive power from the station device and to control charging of a battery, wherein the station device wirelessly supplies power to the power part and controls a water nozzle to supply water to a flowerpot.

In accordance with an aspect, the controller may search for a space, in which the detected illuminance value is equal to or greater than a preset illuminance value, through the autonomous driving and may control the movable flowerpot device to perform rotational motion in the searched space.

In accordance with an aspect, when the illuminance value detected through the solar detection sensor is equal to or less than the preset illuminance value in the searched space, the controller may stop the rotational motion, may search for another space, an illuminance value of which is equal to or greater than the preset illuminance value, through the autonomous driving, and may control the movable flowerpot device to perform the rotational motion in the other searched space.

In accordance with an aspect, the controller may include a real time clock (RTC) module and may determine whether the movable flowerpot device moves to the station device according to time information output from the RTC module.

In accordance with an aspect, the controller may monitor any one of a state of charge of the battery and moisture content of soil in the flowerpot included in the movable flowerpot device and may determine whether the movable flowerpot device moves to the station device according to a monitoring result.

In accordance with an aspect, the power part may include a receiving coil configured to wirelessly receive power, and wherein the station device includes: a transmitting coil configured to wirelessly supply power to the receiving coil, and a detection coil configured to detect misalignment between the transmitting coil and the receiving coil.

In accordance with an aspect, the station device may include the water nozzle configured to supply water to the flowerpot included in the movable flowerpot device; a camera installed in the water nozzle and configured to detect a position of a plant included in the flowerpot; and a nozzle driver configured to adjust a position of the water nozzle based on the position of the plant detected through the camera.

In accordance with yet another aspect of the present invention, there is provided a control method of charging and water supply of a station device of a plant growth management system, the control method comprising: detecting entry of a movable flowerpot device and generating a detection signal according to whether entry of the movable flowerpot device is detected, by a flowerpot detector; wirelessly supplying power to the movable flowerpot device according to the detection signal, by a wireless charger; and supplying water to a flowerpot included in the movable flowerpot device according to the detection signal, by a water supply device, wherein the movable flowerpot device includes an obstacle detection sensor and a solar detection sensor, performs autonomous driving based on the obstacle detection sensor and the solar detection sensor, searches for a space, an illuminance value of which is equal to or greater than a preset illuminance value, through the autonomous driving, and performs rotational motion in the searched space.

In accordance with an aspect, the wirelessly supplying power may further include detecting misalignment between a transmitting coil included in the wireless charger and a receiving coil included in the movable flowerpot device, by a detection coil included in the wireless charger.

In accordance with an aspect, the supplying water to the flowerpot may include detecting a position of a plant included in the flowerpot, by a camera included in a water nozzle; and adjusting a position of the water nozzle based on the detected position of the plant, by a nozzle driver.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a diagram showing a plant growth management system according to an embodiment of the present invention;

FIG. 2 is a diagram showing a station device of a plant growth management system according to an embodiment of the present invention;

FIG. 3 is a diagram showing a movable flowerpot device of a plant growth management system according to an embodiment of the present invention;

FIG. 4 is a diagram showing an example in which a movable flowerpot device performs autonomous driving and rotational motion according to an embodiment of the present invention; and

FIG. 5 is a diagram showing a control method of charging and water supply of a station device according to an embodiment of the present invention;

### DETAILED DESCRIPTION OF THE INVENTION

The present invention will now be described more fully with reference to the accompanying drawings, in which exemplary embodiments of the invention are shown.

This invention, however, should not be construed as limited to the exemplary embodiments and terms used in the exemplary embodiments, and should be understood as including various modifications, equivalents, and substituents of the exemplary embodiments.

Preferred embodiments of the present invention are now described more fully with reference to the accompanying drawings. In the description of embodiments of the present invention, certain detailed explanations of related known functions or constructions are omitted when it is deemed that they may unnecessarily obscure the essence of the invention.

In addition, the terms used in the specification are defined in consideration of functions used in the present invention, and can be changed according to the intent or conventionally used methods of clients, operators, and users. Accordingly, definitions of the terms should be understood on the basis of the entire description of the present specification.

In the drawings, like reference numerals in the drawings denote like elements.

As used herein, the singular forms “a,” “an” and “the” are intended to include the plural forms as well, unless context clearly indicates otherwise.

Expressions such as “A or B” and “at least one of A and/or B” should be understood to include all possible combinations of listed items.

Expressions such as “a first,” “the first,” “a second” and “the second” may qualify corresponding components irrespective of order or importance and may be only used to distinguish one component from another component without being limited to the corresponding components.

In the case in which a (e.g., first) component is referred as “(functionally or communicatively) connected” or “attached” to another (e.g., second) component, the first component may be directly connected to the second component or may be connected to the second component via another component (e.g., third component).

In the specification, the expression “. . . configured to . . . (or set to)” may be used interchangeably, for example, with expressions, such as “. . . suitable for . . . ,” “. . . having ability to . . . ,” “. . . modified to . . . ,” “. . . manufactured to . . . ,” “. . . enabling to . . . ,” or “. . . designed to . . . ,” in the case of hardware or software depending upon situations.

In any situation, the expression “a device configured to . . . ” may refer to a device configured to operate “with another device or component.”

For examples, the expression “a processor configured (or set) to execute A, B, and C” may refer to a specific processor performing a corresponding operation (e.g., embedded processor), or a general-purpose processor (e.g., CPU or application processor) executing one or more software programs stored in a memory device to perform corresponding operations.

In addition, the expression “or” means “inclusive or” rather than “exclusive or”. That is, unless otherwise mentioned or clearly inferred from context, the expression “x uses a or b” means any one of natural inclusive permutations.

That is, unless otherwise mentioned or clearly inferred from context, the expression “x uses a or b” means any one of natural inclusive permutations.

In the aforementioned embodiments, constituents of the present invention were expressed in a singular or plural form depending upon embodiments thereof.

However, the singular or plural expressions should be understood to be suitably selected depending upon a suggested situation for convenience of description, and the aforementioned embodiments should be understood not to be limited to the disclosed singular or plural forms. In other words, it should be understood that plural constituents may be a singular constituent or a singular constituent may be plural constituents.

While the embodiments of the present invention have been described, those skilled in the art will appreciate that many modifications and changes can be made to the present invention without departing from the spirit and essential characteristics of the present invention.

Therefore, it should be understood that there is no intent to limit the disclosure to the embodiments disclosed, rather, the disclosure is to cover all modifications, equivalents, and alternatives falling within the spirit and scope of the disclosure as defined by the claims.

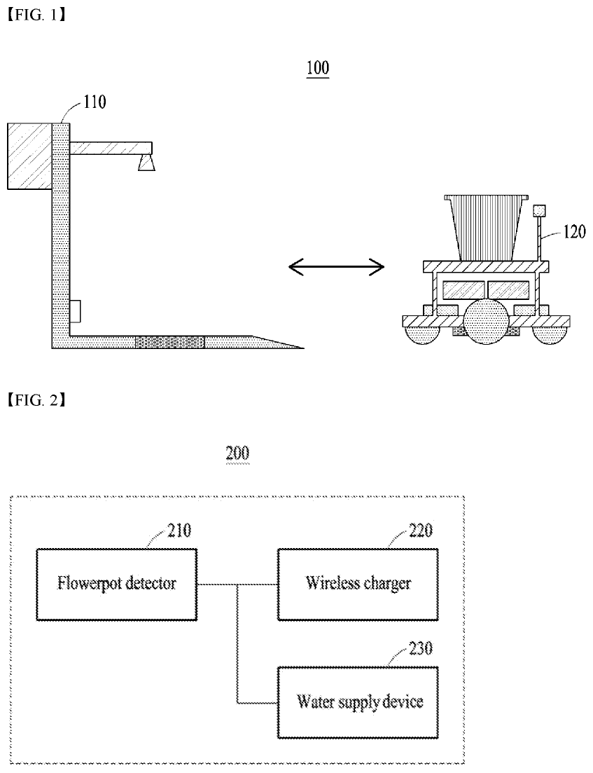

FIG. 1 is a diagram showing a plant growth management system according to an embodiment of the present invention.

Referring to FIG. 1, a plant growth management system 100 according to an embodiment may include a station device 110 and a movable flowerpot device 120.

The station device 110 according to an embodiment may detect entry of the movable flowerpot device 120, may wirelessly supply power to the movable flowerpot device 120 according to the detection result, and may supply moisture to a flowerpot included in the movable flowerpot device 120.

The movable flowerpot device 120 according to an embodiment may perform autonomous driving based on an obstacle detection sensor and a solar detection sensor, may search for a space, illuminance value of which is equal to or greater than a preset illuminance value, through autonomous driving, may perform rotational motion in the searched space, and may determine whether to move to the station device 110.

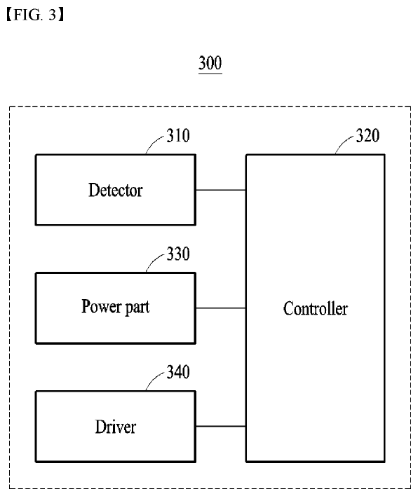

The configuration and operation of the station device 110 and the movable flowerpot device 120 according to an embodiment will be described below in more detail with reference to FIGS. 2 to 4 showing other embodiments.

FIG. 2 is a diagram showing a station device of a plant growth management system according to an embodiment of the present invention.

Referring to FIG. 2, a station device 200 according to an embodiment may detect entry of a movable flowerpot device, may wirelessly supply power to the movable flowerpot device according to the detection result, and may supply moisture to a flowerpot included in the movable flowerpot device.

To this end, the station device 200 may include a flowerpot detector 210, a wireless charger 220, and a water supply device 230.

The flowerpot detector 210 according to an embodiment may detect entry of the movable flowerpot device and may generate a detection signal according to whether entry of the movable flowerpot device is detected.

In detail, the flowerpot detector 210 may detect the movable flowerpot device that enters the station device 200 through an arrival detection sensor included in the station device 200 and may generate the detection signal upon detecting entry of the movable flowerpot device.

For example, the arrival detection sensor may include any one of a distance sensor and an ultrasonic sensor, but a type of the arrival detection sensor is not limited thereto.

The flowerpot detector 210 may recognize a proximity degree of the movable flowerpot device and may also transfer a stop command to the movable flowerpot device when the movable flowerpot device approaches the station device 200.

The movable flowerpot device according to an embodiment may include an obstacle detection sensor and a solar detection sensor, may perform autonomous driving based on an obstacle detection sensor and a solar detection sensor, may search for a space, illuminance value of which is equal to or greater than a preset illuminance value, through autonomous driving, and may perform rotational motion in the searched space.

According to an aspect, the movable flowerpot device may determine whether to move to the station device according to time information output from a real time clock (RTC) module.

The movable flowerpot device may monitor at least one of a state of charge (SoC) of a battery or a moisture content of the soil in the flowerpot and may also determine whether to move to the station device according to the monitoring result.

When the illuminance value detected through the solar detection sensor is equal to or less than a preset illuminance value in the searched space, the movable flowerpot device may stop rotational motion, may search for another space, illuminance value of which is equal to or greater than a preset illuminance value, through autonomous driving, and may perform rotational motion in the other searched space.

The configuration and operation of the movable flowerpot device according to an embodiment will be described below in more detail with reference to FIG. 3 showing an embodiment.

The wireless charger 220 according to an embodiment may wirelessly supply power to the movable flowerpot device according to the detection signal generated by the flowerpot detector 210.

In detail, upon receiving the detection signal, the wireless charger 220 may wirelessly transmit power to a receiving coil included in the movable flowerpot device that enters the station device 200.

According to an aspect, the wireless charger 220 may include a transmitting coil for wirelessly transmitting power to the receiving coil included in the movable flowerpot device, and a detection coil for detecting misalignment between the transmitting coil and the receiving coil.

In detail, upon detecting misalignment between the transmitting coil and the receiving coil through the detection coil, the wireless charger 220 may generate a position control signal for controlling the position of the movable flowerpot device to accurately align the transmitting coil and the receiving coil.

For example, the wireless charger 220 may detect a wireless power transfer rate between the transmitting coil and the receiving coil through the detection coil, and may generate the position control signal for performing control to move the position of the movable flowerpot device to a position at which the detected wireless power transfer rate is the maximum.

That is, according to the present invention, the transmitting coil included in the station device 200 and the receiving coil included in the movable flowerpot device may be accurately aligned, thereby improving charging efficiency and reducing a charging time.

The water supply device 230 according to an embodiment may supply water to a flowerpot included in the movable flowerpot device according to the detection signal generated by the flowerpot detector 210.

In detail, upon receiving the detection signal, the water supply device 230 may control an operation of a water nozzle to supply water to the flowerpot included in the movable flowerpot device.

For example, upon receiving the detection signal, the water supply device 230 may supply water to the flowerpot according to the number and amount set by the user.

According to an aspect, the water supply device 230 may include a water nozzle for supplying water to the flowerpot, a camera installed in the water nozzle and configured to detect the position of a plant in the flowerpot, and a nozzle driver for adjusting the position of the water nozzle based on the position of the plant detected through the camera.

In detail, the water supply device 230 may capture an image of a top of the flowerpot through the camera included in the water nozzle and may distinguish between a region in which a plant is present and a soil region in which a plant is not present in the captured image of the top of the flowerpot.

Then, the water supply device 230 may adjust the position of the water nozzle in order to selectively supply water to any one of the region in which a plant is present and the soil region according to input information.

For example, the water supply device 230 may supply water to a desired position by adjusting the direction and height of the water nozzle through the nozzle driver.

In general, depending on a plant type, when water is supplied directly from the top of flower, the flower and leaf may be damaged and a bulb may rot, and in the case of this plant type, water is not supplied directly to the plant, but instead, the plant needs to be watered to allow water to seep only in roots, and thus the position of the water nozzle needs to be controlled.

To overcome this, the water supply device 230 according to an embodiment may selectively supply water according to input information, thereby minimizing damage to plants due to water supply and inducing successful growth.

For example, the input information may be information input for a user to directly select any one of the region in which a plant is present and the soil region.

When the water supply device 230 is connected to the Internet of Things (IoT), the input information may be input from an external server.

In more detail, upon receiving the information input from the external server, the water supply device 230 may identify a plant type by comparing image information included in the information input from the external server with image information of a plant photographed by the camera, may select any one of the region in which a plant is present and the soil region in consideration of the characteristics of the identified plant type, and may supply water.

For example, the water supply device 230 may extract the shape of any one of shapes of a flower, a leave, and a stem included in the plant from image information of the plant photographed by the camera, and may identify a plant type by comparing the extracted shape with the image information included in the information input from the external server.

That is, when the station device according to the present invention is used, wireless charging and automatic water supply may be provided to the movable flowerpot device, thereby stably supplying power and inducing successful growth of plants.

FIG. 3 is a diagram showing a movable flowerpot device of a plant growth management system according to an embodiment of the present invention.

Among the contents described through the embodiment of FIG. 3, redundant descriptions of those described through the movable flowerpot device of FIGS. 1 and 2 will be omitted.

Referring to FIG. 3, a movable flowerpot device 300 according to an embodiment may detect presence of an obstacle and a surrounding illuminance value, may perform control to perform autonomous driving according to the detection result, and may determine whether to move to the station device.

To this end, the movable flowerpot device 300 according to an embodiment may include a detector 310, a controller 320, and a power part 330.

The movable flowerpot device 300 according to an embodiment may further include a driver 340 for controlling movement of the movable flowerpot device 300 according to a control operation of the controller 320.

In detail, the driver 340 may include a plurality of wheels and a plurality of motors that are operatively associated with the plurality of wheels.

The detector 310 according to an embodiment may include an obstacle detection sensor and a solar detection sensor, may detect presence of an obstacle through the obstacle detection sensor, and may detect an illuminance value through the solar detection sensor.

For example, the obstacle detection sensor may include any one or more of a distance sensor and an ultrasonic sensor, and the solar detection sensor may include an illuminance sensor, but a type of a sensor included in the detector 310 is limited thereto.

The obstacle detection sensors may be installed at both ends of a front surface of the movable flowerpot device 300 to detect a distance between an obstacle and the movable flowerpot device 300 and the movable flowerpot device 300 may prevent collision with the obstacle according to the detected distance.

The controller 320 according to an embodiment may determine a moving path based on whether an obstacle is present and the detected illuminance value, may control the movable flowerpot device 300 to perform autonomous driving on the determined moving path, may determine whether the movable flowerpot device 300 moves to the station device, and may control the movable flowerpot device 300 to move to the station device according to the determination result of whether the movable flowerpot device 300 moves to the station device.

According to an aspect, the controller 320 may search for a space, illuminance value of which is equal to or greater than a preset illuminance value, through autonomous driving, and may perform rotational motion in the searched space.

In detail, the controller 320 may receive information on whether an obstacle is present and the detected illuminance value from the detector 310 in real time, may determine a moving path to move to a space illuminated by sunlight based on the information received in real time, and may control the movable flowerpot device 300 to perform autonomous driving on the determined moving path.

Then, when the movable flowerpot device 300 reaches a space in which the illuminance value received in real time is equal to or greater than a preset illuminance value during autonomous driving, the controller 320 may stop autonomous driving and may control the movable flowerpot device 300 to perform rotational motion in place.

For example, a preset illuminance value may be an initial value set by the movable flowerpot device in order to identify the space illuminated by sunlight and may also be set by a user.

That is, the movable flowerpot device 300 according to the present invention may search for the space illuminated by sunlight to move thereto, and may perform rotational motion in the space illuminated by sunlight to allow plants to uniformly absorb sunlight in all directions, thereby inducing successful growth of plants.

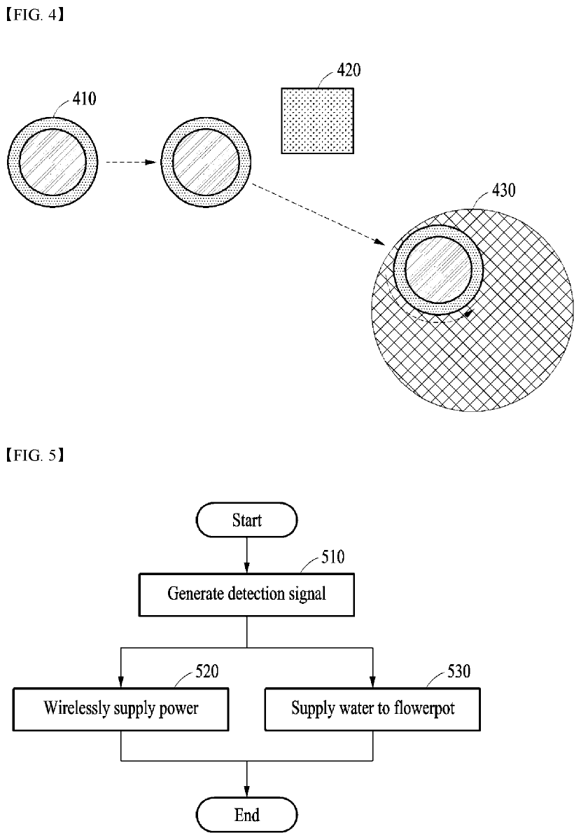

The configuration of performing autonomous driving and rotational motion in the movable flowerpot device 300 according to an embodiment will be described below in more detail with reference to FIG. 4 showing an embodiment.

According to an aspect, when an illuminance value detected through the solar detection sensor in the searched space is equal to or less than a preset illuminance value, the controller 320 may stop rotational motion, may search for another space, illuminance value of which is equal to or greater than a preset illuminance value, through autonomous driving, and may perform rotational motion in the other searched space.

In other words, when a space illuminated by sunlight is changed over time, the controller 320 may control the movable flowerpot device 300 to re-perform autonomous driving in order to re-search for the changed space illuminated by sunlight and may control the movable flowerpot device 300 to perform rotational motion in the changed space illuminated by sunlight through re-performed autonomous driving.

That is, the controller 320 according to the present invention may track the space illuminated by sunlight, which is changed over time, and may control the movable flowerpot device 300 to move along the changed space illuminated by sunlight, thereby improving solar absorption time and efficiency of plants.

According to an aspect, the controller 320 may include a real time clock (RTC) module and may determine whether the movable flowerpot device 300 moves to the station device according to time information output from the RTC module.

In detail, the controller 320 may control plants to absorb sunlight during the daytime based on current time information output from the real time clock (RTC) module and may control the movable flowerpot device 300 to return to the station device during the evening when the sun goes down.

For example, the controller 320 may pre-store information on the time to leave the station device for absorption of sunlight and the time to return to the station device.

According to an aspect, the controller 320 may monitor any one of a state of charge of a battery or moisture content of the soil in the flowerpot included in the movable flowerpot device and may determine whether the movable flowerpot device moves to the station device according to the monitoring result.

In detail, the movable flowerpot device 300 may include a device for detecting the state of charge of the battery and a sensor for monitoring moisture content of the soil in the flowerpot, and as such, the controller 320 may monitor the state of charge of the battery and the moisture content of the soil.

Then, when the state of charge of the charge or the moisture content of the soil is equal to or less than a preset value, the controller 320 may determine the movable flowerpot device 300 to return to the station device.

The power part 330 according to an embodiment may wirelessly receive power from the station device and may control charging of the battery.

The station device according to an embodiment may wirelessly supply power to the power part 330 and may control the water nozzle to supply water to the flowerpot.

According to an aspect, the power part 330 may include a receiving coil for wirelessly receiving power, and the station device may include a transmitting coil for wirelessly supplying power, and a detection coil for detecting misalignment between the transmitting coil and the receiving coil.

According to an aspect, the station device may include a water nozzle for supplying water to the flowerpot included in the movable flowerpot device 300, a camera installed in the water nozzle and configured to detect the position of a plant in the flowerpot, and a nozzle driver for adjusting the position of the water nozzle based on the position of the plant detected through the camera.

That is, the movable flowerpot device 300 according to the present invention may autonomously change the position of the flowerpot, and thus may automatically adjust the amount of sunlight that a plant is capable of receiving and may prevent damage to the plant due to movement of the flowerpot, thereby improving user convenience.

FIG. 4 is a diagram showing an example in which a movable flowerpot device performs autonomous driving and rotational motion according to an embodiment of the present invention.

Referring to FIG. 4, reference numeral 410 denotes the movable flowerpot device 300 of FIG. 3, reference numeral 420 denotes an obstacle present on a moving path, and reference numeral 430 denotes a space, an illuminance value of which is equal to or greater than a preset illuminance value.

First, a movable flowerpot device 410 may determine a moving path for autonomous driving based on whether an obstacle detected through an obstacle detection sensor is present and an illuminance value detected through a solar detection sensor.

In detail, a controller of the movable flowerpot device 410 may be embodied as the Arduino Uno and may control a plurality of motors included in the driver 340 of FIG. according to the detection results of the obstacle detection sensor and the solar detection sensor to control movement of the movable flowerpot device 410.

In more detail, the controller of the movable flowerpot device 410 may inform a rotation angle to a plurality of motors to correct errors that occur while the plurality of motors operate and to measure a rotation angle during rotational motion.

The controller of the movable flowerpot device 410 may control the movable flowerpot device 410 to continuously move in a direction in which the device proceeds after rotational motion is stopped, and when an obstacle needs to be avoided or rotational motion is required, the controller may control the movable flowerpot device 410 to move after rotating at an accurate angle.

The movable flowerpot device 410 may detect presence of an obstacle 420 present on a moving path through the obstacle detection sensor during autonomous driving and may re-correct the moving path not to collide with the detected obstacle 420.

The movable flowerpot device 410 may search for a space 430 in which an illuminance value detected in real time through a solar detection sensor is equal to or greater than a preset illuminance value, while autonomously driving on the moving path, and when the movable flowerpot device 410 reaches the searched space 430, the illuminance value is equal to or greater than a preset illuminance value, the movable flowerpot device 410 may stop autonomous driving and may perform rotational motion to allow plants included in the movable flowerpot device 410 to uniformly absorb sunlight in all directions.

FIG. 5 is a diagram showing a control method of charging and water supply of a station device according to an embodiment of the present invention.

An embodiment of FIG. 5 relates to a control method of charging and water supply using the station device of FIG. 2, and hereinafter, among the contents described through the embodiment of FIG. 5, redundant descriptions of those described through the station device of FIG. 2 will be omitted.

Referring to FIG. 5, in operation 510 of the control method of charging and water supply according to an embodiment, a flowerpot detector may detect entry of a movable flowerpot device and may generate a detection signal according to the detection result.

According to an aspect, the movable flowerpot device may include an obstacle detection sensor and a solar detection sensor, may perform autonomous driving based on the obstacle detection sensor and the solar detection sensor, may search for a space, an illumination value is equal to or greater than a preset illuminance value, through autonomous driving, and may perform rotational motion in the searched space.

In operation 520 of the control method of charging and water supply according to an embodiment, a wireless charger may wirelessly supply power to the movable flowerpot device according to a detection signal.

According to an aspect, operation 520 in the control method of charging and water supply according to an embodiment may further include detecting misalignment between a transmitting coil included in the wireless charger and a receiving coil included in the movable flowerpot device by a detection coil included in the wireless charger.

In operation 530 of the control method of charging and water supply according to an embodiment, a water supply device may supply water to a flowerpot included in the movable flowerpot device according to the detection signal.

According to an aspect, operation 530 of the control method of charging and water supply according to an embodiment may further include detecting the position of a plan included in a flowerpot by a camera installed in the water nozzle and adjusting the position of the water nozzle based on the detected position of the plant by a nozzle driver.

As a result, according to the present invention, the position of a flowerpot may be autonomously changed, and thus the amount of sunlight that a plant is capable of receiving may be automatically adjusted, and damage to the plants due to movement of the flowerpot may be prevented, thereby improving user convenience.

The present invention may provide wireless charging and automatic water supply, thereby stably supplying power and inducing successful growth of plants.

According to the present invention, water may be selectively supplied to a flowerpot, thereby minimizing damage to plants due to moisture supply.

According to an embodiment, the position of a flowerpot may be autonomously changed, and thus the amount of sunlight that a plant is capable of receiving may be autonomously adjusted, and damage to the plant due to movement of the flowerpot may be prevented, thereby improving user convenience.

According to an embodiment, wireless charging and automatic water supply may be provided, thereby stably supplying power and inducing successful growth of plants.

According to an embodiment, water may be selectively supplied to a flowerpot, thereby minimizing damage to plants due to moisture supply.

The apparatus described above may be implemented as a hardware component, a software component, and/or a combination of hardware components and software components. For example, the apparatus and components described in the embodiments may be achieved using one or more general purpose or special purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of executing and responding to instructions. The processing device may execute an operating system (OS) and one or more software applications executing on the operating system. In addition, the processing device may access, store, manipulate, process, and generate data in response to execution of the software. For ease of understanding, the processing apparatus may be described as being used singly, but those skilled in the art will recognize that the processing apparatus may include a plurality of processing elements and/or a plurality of types of processing elements. For example, the processing apparatus may include a plurality of processors or one processor and one controller. Other processing configurations, such as a parallel processor, are also possible.

The software may include computer programs, code, instructions, or a combination of one or more of the foregoing, configure the processing apparatus to operate as desired, or command the processing apparatus, either independently or collectively. In order to be interpreted by a processing device or to provide instructions or data to a processing device, the software and/or data may be embodied permanently or temporarily in any type of a machine, a component, a physical device, a virtual device, a computer storage medium or device, or a transmission signal wave.

The software may be distributed over a networked computer system and stored or executed in a distributed manner. The software and data may be stored in one or more computer-readable recording media.

The methods according to the embodiments of the present disclosure may be implemented in the form of a program command that can be executed through various computer means and recorded in a computer-readable medium. The computer-readable medium can store program commands, data files, data structures or combinations thereof. The program commands recorded in the medium may be specially designed and configured for the present disclosure or be known to those skilled in the field of computer software. Examples of a computer-readable recording medium include magnetic media such as hard disks, floppy disks and magnetic tapes, optical media such as CD-ROMs and DVDs, magneto-optical media such as floptical disks, or hardware devices such as ROMs, RAMs and flash memories, which are specially configured to store and execute program commands. Examples of the program commands include machine language code created by a compiler and high-level language code executable by a computer using an interpreter and the like. The hardware devices described above may be configured to operate as one or more software modules to perform the operations of the embodiments, and vice versa.

### DESCRIPTION OF REFERENCE NUMERALS

| | |
|---|---|
| 200: station device | 210: flowerpot detector |
| 220: wireless charger | 230: water supply device |

What is claimed is:

1. A station device of a plant growth management system, comprising:
a flowerpot detector configured detect entry of a movable flowerpot device and to generate a detection signal according to whether entry of the movable flowerpot device is detected;
a wireless charger configured to wirelessly supply power to the movable flowerpot device according to the detection signal; and
a water supply device configured to supply water to a flowerpot included in the movable flowerpot device according to the detection signal,
wherein the movable flowerpot device includes an obstacle detection sensor and a solar detection sensor, performs autonomous driving based on the obstacle detection sensor and the solar detection sensor, searches for a space, an illuminance value of which is equal to or greater than a preset illuminance value, through the autonomous driving, and performs rotational motion in the searched space.

2. The station device of the plant growth management system according to claim 1, wherein the wireless charger includes:
a transmitting coil configured to wirelessly supply power to a receiving coil included in the movable flowerpot device; and
a detection coil configured to detect misalignment between the transmitting coil and the receiving coil.

3. The station device of the plant growth management system according to claim 1, wherein the water supply device includes:
a water nozzle configured to supply water to the flowerpot;
a camera installed in the water nozzle and configured to detect a position of a plant included in the flowerpot; and
a nozzle driver configured to adjust a position of the water nozzle based on the position of the plant detected through the camera.

4. The station device of the plant growth management system according to claim 1, wherein the movable flowerpot device determines whether to move to the station device according to time information output from a real time clock (RTC) module.

5. The station device of the plant growth management system according to claim 1, wherein the movable flowerpot device monitors at least one of a state of charge of a battery and moisture content of soil in the flowerpot and determines whether to move to the station device according to a monitoring result.

6. The station device of the plant growth management system according to claim 1, wherein, when an illuminance value detected through the solar detection sensor is equal to or less than the preset illuminance value in the searched space, the movable flowerpot device stops the rotational motion, searches for another space, an illuminance value of which is equal to or greater than the preset illuminance value, through the autonomous driving, and performs the rotational motion in the other searched space.

7. A movable flowerpot device of a plant growth management system, comprising:
a detector including an obstacle detection sensor and a solar detection sensor, and configured to detect whether an obstacle is present through the obstacle detection sensor and to detect an illuminance value through the solar detection sensor;
a controller configured to determine a moving path based on whether the obstacle is present and the detected illuminance value, to control the movable flowerpot device to perform autonomous driving on the moving path, to determine whether the movable flowerpot device moves to a station device, and to control the movable flowerpot device to move to the station device according to a determination result of whether the movable flowerpot device moves to the station device; and
a power part configured to wirelessly receive power from the station device and to control charging of a battery,
wherein the station device wirelessly supplies power to the power part and controls a water nozzle to supply water to a flowerpot.

8. The movable flowerpot device of the plant growth management system according to claim 7, wherein the controller searches for a space, in which the detected illuminance value is equal to or greater than a preset illuminance value, through the autonomous driving and controls the movable flowerpot device to perform rotational motion in the searched space.

9. The movable flowerpot device of the plant growth management system according to claim 8, wherein, when the illuminance value detected through the solar detection sensor is equal to or less than the preset illuminance value in the searched space, the controller stops the rotational motion, searches for another space, an illuminance value of which is equal to or greater than the preset illuminance value, through the autonomous driving, and controls the movable flowerpot device to perform the rotational motion in the other searched space.

10. The movable flowerpot device of the plant growth management system according to claim 7, wherein the controller includes a real time clock (RTC) module and determines whether the movable flowerpot device moves to the station device according to time information output from the RTC module.

11. The movable flowerpot device of the plant growth management system according to claim 7, wherein the controller monitors any one of a state of charge of the battery and moisture content of soil in the flowerpot included in the movable flowerpot device and determines whether the movable flowerpot device moves to the station device according to a monitoring result.

12. The movable flowerpot device of the plant growth management system according to claim 7, wherein the power part includes a receiving coil configured to wirelessly receive power, and
wherein the station device includes:
a transmitting coil configured to wirelessly supply power to the receiving coil, and a detection coil configured to detect misalignment between the transmitting coil and the receiving coil.

13. The movable flowerpot device of the plant growth management system according to claim 7, wherein the station device includes:
the water nozzle configured to supply water to the flowerpot included in the movable flowerpot device;
a camera installed in the water nozzle and configured to detect a position of a plant included in the flowerpot; and
a nozzle driver configured to adjust a position of the water nozzle based on the position of the plant detected through the camera.

14. A control method of charging and water supply of a station device of a plant growth management system, the control method comprising:
detecting entry of a movable flowerpot device and generating a detection signal according to whether entry of the movable flowerpot device is detected, by a flowerpot detector;
wirelessly supplying power to the movable flowerpot device according to the detection signal, by a wireless charger; and
supplying water to a flowerpot included in the movable flowerpot device according to the detection signal, by a water supply device,
wherein the movable flowerpot device includes an obstacle detection sensor and a solar detection sensor, performs autonomous driving based on the obstacle detection sensor and the solar detection sensor, searches for a space, an illuminance value of which is equal to or greater than a preset illuminance value, through the autonomous driving, and performs rotational motion in the searched space.

15. The control method according to claim 14, wherein the wirelessly supplying power further includes:
detecting misalignment between a transmitting coil included in the wireless charger and a receiving coil included in the movable flowerpot device, by a detection coil included in the wireless charger.

16. The control method of claim 14, wherein the supplying water to the flowerpot includes:
detecting a position of a plant included in the flowerpot, by a camera included in a water nozzle; and
adjusting a position of the water nozzle based on the detected position of the plant, by a nozzle driver.
